(19) United States
(12) Patent Application Publication
(10) Pub. No.: US 2023/0173013 A1
(43) Pub. Date: Jun. 8, 2023

(54) FOOD FOR HANGOVER AND LIVER PROTECTION AND PRODUCING METHOD THEREOF

(71) Applicant: Meyer Health Jiangsu Co., Ltd., Taizhou (CN)

(72) Inventors: Hanping SHI, Taizhou (CN); Ru JIA, Taizhou (CN)

(21) Appl. No.: 17/540,244

(22) Filed: Dec. 2, 2021

(51) Int. Cl. A61K 36/9066; A61K 31/355; A23L 33/00; A61K 31/525; A61K 36/185; A23L 33/15; A23L 33/105; A61K 36/488; A61P 1/16; A61K 31/375; A61K 31/4415; A61K 31/51; A61K 47/46

(52) U.S. Cl. CPC A61K 36/9066; A23L 33/15; A23L 33/40; A23L 33/105; A61K 31/51; A61K 31/355; A61K 31/375; A61K 31/525; A61K 31/4415; A61K 36/185; A61K 36/488; A61K 47/46; A61P 1/16; A23V 2002/00

(57) ABSTRACT

The present disclosure provides a food for hangover and liver protection and a producing method thereof. The method includes: providing the raw materials of particular percentages by weight, wherein the prepared turmeric powder accounts for 25%-35% of the provided raw materials; the prepared kudzu isoflavone powder accounts for 30%-40% of the provided raw materials; the prepared Primula sieboldii root powder accounts for 20%-30% of the provided raw materials; the prepared fruit extract accounts for 5%-14% of the provided raw materials; and the prepared vitamin complex accounts for 1%-2% of the provided raw materials; performing a sieving pretreatment on each of the provided raw materials; putting the pretreated raw materials into a wet granulator to perform a mix process for obtaining the food; and performing a testing process on the mixed food, and re-performing the mix process on the tested food in response to the tested food being unqualified.

### BACKGROUND

### 1. Technical Field

The present disclosure relates to health food technology, and particularly to a food for hangover and liver protection and a producing method thereof.

### 2. Description of Related Art

With the improvement of people’s living standards and the increasement of the frequency of various social activities, alcohol has become an indispensable drink in the daily life and various social activities. Although drinking alcohol in moderation is good for health, excessive drinking will not only produce a series of uncomfortable symptoms of alcoholism, but also cause great damage to the liver of human body.

After entering the human body, about 90% of alcohol will be metabolized by the liver. This metabolism mainly relies on two enzymes in the liver’s enzyme system, namely alcohol dehydrogenase and acetaldehyde dehydrogenase. The alcohol is converted into acetaldehyde by alcohol dehydrogenase first, then the converted acetaldehyde is converted into acetic acid by acetaldehyde dehydrogenase, and finally decomposed into adenosine triphosphate, water and carbon dioxide to excrete, thereby completely achieving the metabolic process. There is alcohol dehydrogenase in every normal human body and its amount is roughly the same, but the amount of acetaldehyde dehydrogenase in most human bodies is insufficient. Due to the lack of acetaldehyde dehydrogenase, the acetaldehyde converted from alcohol cannot be completely decomposed into acetic acid, and will remain in the liver of the human body, which increases the burden on the liver and at the same time causes the human to have nausea, vomiting, coma, and other drunken symptoms. On the other hand, if a normal human drinks too much or too fast at one time which exceeds the metabolic capacity of acetaldehyde dehydrogenase in the human body, drunkenness will also occur. Therefore, enhancing the activities of the related alcohol-metabolizing enzymes such as alcohol dehydrogenase (ADH) and acetaldehyde dehydrogenase (ALDH) in the liver is the main pharmacological basis for the existing hangover products.

At present, most of the hangover products on the market are the drinks made from complex Chinese medicinal materials or quick hangover drugs. These Chinese medicinal materials have complex prescriptions and a few extracted effective ingredients, so the effect is slow and the amount required is large while the liver protection effect is not obvious. The quick hangover drugs have large side effects and poor safety, which can only temporarily relieve the hangover phenomenon and cannot repair the liver damage caused by alcohol allergy or excessive drinking, and is incapable of protecting the liver and prevent it from damage.

### SUMMARY

In view of the above-mentioned problems, the present disclosure provides a food for hangover and liver protection. The quality ratio of raw materials of the food for hangover and liver protection includes: 25%-35% of turmeric powder; 30%-40% of kudzu isoflavone powder; 20%-30% of Primula sieboldii root powder; 5%-14% of fruit extract; and 1%-2% of vitamin complex.

Furthermore, the turmeric powder is nano turmeric powder.

Furthermore, the kudzu isoflavone powder includes one or two of 10% of isoflavone, 20% of isoflavone, and 40% of isoflavone.

Furthermore, the Primula sieboldii root powder is ground powder of Primula sieboldii root.

Furthermore, the fruit extract includes one or more of extracts of apple, guava, persimmon, and citrus peel.

Furthermore, the vitamin complex includes at least two of sodium L-ascorbate, dl-α-tocopheryl acetate, pyridoxine hydrochloride, riboflavin, thiamine hydrochloride, and thiamine nitrate.

The present disclosure further provides a producing method for a food for hangover and liver protection, which includes:
preparing raw materials of the food, where the raw materials include turmeric powder, kudzu isoflavone powder, Primula sieboldii root powder, fruit extract, and vitamin complex;
providing the raw materials of particular percentages by weight, where the prepared turmeric powder accounts for 25%-35% of the provided raw materials; the prepared kudzu isoflavone powder accounts for 30%-40% of the provided raw materials; the prepared Primula sieboldii root powder accounts for 20%-30% of the provided raw materials; the prepared fruit extract accounts for 5%-14% of the provided raw materials; and the prepared vitamin complex accounts for 1%-2% of the provided raw materials;
performing a sieving pretreatment on each of the provided raw materials;
putting the pretreated raw materials into a wet granulator to perform a mix process for obtaining the food; and
performing a testing process on the mixed food, and re-performing the mix process on the tested food in response to the tested food being unqualified.

Furthermore, the turmeric powder is prepared by providing turmeric to grind into coarse powder and add ethanol aqueous solution of 5-8 times of the weight of the turmeric and mass fraction of 70-80% to stir and let stand for 48 hours, providing a supernatant to vacuum concentrate to a saturated solution and let stand for cooling so as to precipitate a solid, and adding the solid after drying to an aqueous solution containing the surfactant Tween 80 and spray-drying through high pressure homogenize to obtain nano-turmeric powder with a diameter of 100-1000 nanometers;
the kudzu isoflavone powder is prepared by providing Pueraria root to grind into coarse powder and add distilled water of 5-10 times of the weight of the Pueraria root to stir for 0.5-1 hour and filter to obtain filtrate, repeating the providing Primula sieboldii root for five times to combine the obtained filtrate, letting stand the combined filtrate for 1-2 days at 25-30° C. to obtain a precipitate, and filtering and drying the obtained precipitate to grind into fine powder as the kudzu isoflavone powder; and
the Primula sieboldii root powder is prepared by providing Primula sieboldii root to grind into coarse powder and put into a juicer with 100-200 meshes to squeeze to obtain Primula sieboldii root juice, drying the Primula sieboldii root juice in a drying box at 60-80° C. to obtain a dried solid, and putting the dried solid in a pulverizer for pulverizing as the Primula sieboldii root powder.

Furthermore, the fruit extract is prepared by providing fresh fruit to wash and chop and then add ethanol aqueous solution of 5-10 times of the weight of the fruit and mass fraction of 70-80% to fully stir and add pectinase with mass fraction of 0.05% to perform enzymatic hydrolysis for 5-8 hours in 40-50° C. to obtain a enzymatic hydrolysis solution, and heating the enzymatic hydrolysis solution to 100° C. for inactivating the pectinase and centrifuging at 5000 rpm in 25° C. for 20 minutes to obtain a supernatant, drying the obtained supernatant at 30-40° C. to freeze-dry as the fruit extract.

Furthermore, a sieve is used for sieving in the sieving pretreatment, and the sieve has 40 or 60 meshes.

Furthermore, the mix process includes:

adjusting a mixing time according to the total weight of the raw materials.

Furthermore, the mix process further includes:

obtaining the food by pouring the pretreated turmeric powder, kudzu isoflavone powder, Primula sieboldii root powder, fruit extract and vitamin complex into a mixing unit of the wet granulator for mixing.

Furthermore, the testing process includes:

observing whether the food contains any clump, and determining the food as needing re-performing the mix process in response to the food containing any clump.

Furthermore, the testing process further includes:

determining the mixed food as qualified for posting with a material label and putting into a warehouse for inspection and releasing, in response to the mixed food not containing any clump.

The beneficial effects of the present disclosure include:
1. The food for hangover and liver protection provided by the present disclosure contains kudzu root isoflavone powder, turmeric powder and Primula sieboldii root powder. Experiments have shown that kudzu powder and turmeric powder have a certain promotion effect on activating alcohol dehydrogenase and acetaldehyde dehydrogenase, while Primula sieboldii root powder has almost no effect on activating alcohol dehydrogenase and acetaldehyde dehydrogenase. However, when these compositions are used in combination, the activation rate of alcohol dehydrogenase and acetaldehyde dehydrogenase can be significantly improved, which indicates that the medicinal compositions in kudzu powder, turmeric powder and Primula sieboldii root powder can play a synergistic improvement effect and greatly enhance the activity of alcohol dehydrogenase and acetaldehyde dehydrogenase in the human body. In addition, when these three ingredients are used at the same time, the synergistic improvement effect is the most significant, which can quickly promote the alcohol metabolism in the human body. The hangover and liver protection effect are good and quick, and there are no other adverse side effects after consumption.
2. The food for hangover and liver protection provided by the present disclosure has simple compositions, low cost, simple production process, mildness, stability, and safety, and has great potential for subsequent industrialized production and large promotion possibility.

Other features and advantages of the present disclosure will be described in the following specification, and will partly become obvious from the specification or be understood by implementing the present disclosure. The purpose and other advantages of the present disclosure can be implemented and obtained through the structures indicated in the specification, claims and drawings.

### BRIEF DESCRIPTION OF THE DRAWINGS

To describe the technical schemes in the embodiments of the present disclosure or in the prior art more clearly, the following briefly introduces the drawings required for describing the embodiments or the prior art. It should be noted that, the drawings in the following description merely show some embodiments. For those skilled in the art, other drawings may be obtained according to the drawings without creative efforts.

FIG. 1 is a flow chart of a producing method for a food for hangover and liver protection according to an embodiment of the present disclosure.

### DETAILED DESCRIPTION

In order to make the objects, technical solutions and advantages of the embodiments of the present disclosure more clear, the technical solutions of the embodiments of the present disclosure will be clearly and completely described below with reference to the drawings in the embodiments of the present disclosure. Apparently, the described embodiments are part of the embodiments of the present disclosure, not all of the embodiments. All other embodiments obtained by those skilled in the art based on the embodiments of the present disclosure without creative efforts are within the scope of the present disclosure.

In some embodiments of the present disclosure, a food for hangover and liver protection is provided. The quality ratio of raw materials of the food for hangover and liver protection includes: 25%-35% of turmeric powder; 30%-40% of kudzu isoflavone powder; 20%-30% of Primula sieboldii root powder; 5%-14% of fruit extract; and 1%-2% of vitamin complex.

Furthermore, the turmeric powder is nano turmeric powder.

Furthermore, the kudzu isoflavone powder includes one or two of 10% of isoflavone, 20% of isoflavone, and 40% of isoflavone.

Furthermore, the Primula sieboldii root powder is ground powder of Primula sieboldii root.

Furthermore, the fruit extract includes one or more of extracts of apple, guava, persimmon, and citrus peel.

Furthermore, the vitamin complex includes at least two of sodium L-ascorbate, dl-α-tocopheryl acetate, pyridoxine hydrochloride, riboflavin, thiamine hydrochloride, and thiamine nitrate.

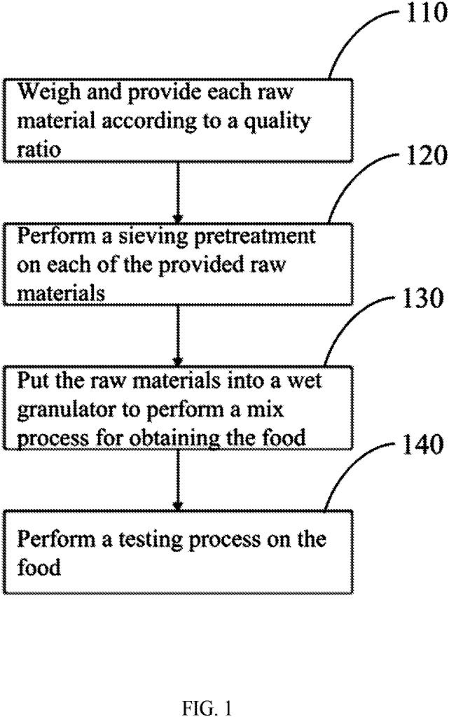

In some embodiments of the present disclosure, a producing method for a food for hangover and liver protection is further provided. FIG. 1 is a flow chart of a producing method for a food for hangover and liver protection according to an embodiment of the present disclosure. The producing method includes:

preparing raw materials;
110: weighing and providing each raw material according to a quality ratio;
120: performing a sieving pretreatment on each raw material;
130: putting the raw materials into a wet granulator to perform a mix process for obtaining the food; and
140: performing a testing process on the well-mixed food.

Specifically, the weighing and providing each raw material includes:

Based on quality ratios, weighing and providing 25%-35% of turmeric powder, 30%-40% of kudzu isoflavone powder, 20%-30% of Primula sieboldii root powder, 5%-14% of fruit extract, and 1%-2% of vitamin complex.

Furthermore, the turmeric powder is nano turmeric powder;
the kudzu isoflavone powder includes one or two of 10% of isoflavone, 20% of isoflavone, and 40% of isoflavone;
the Primula sieboldii root powder is ground powder of Primula sieboldii root;
the fruit extract includes one or more of extracts of apple, guava, persimmon, and citrus peel; and
the vitamin complex includes at least two of sodium L-ascorbate, dl-α-tocopheryl acetate, pyridoxine hydrochloride, riboflavin, thiamine hydrochloride, and thiamine nitrate.

Furthermore, the turmeric powder is prepared by providing turmeric to grind into coarse powder and add ethanol aqueous solution of 5-8 times of the weight of the turmeric and mass fraction of 70-80% to stir and let stand for 48 hours, providing a supernatant to vacuum concentrate to a saturated solution and let stand for cooling so as to precipitate a solid, and adding the solid after drying to an aqueous solution containing the surfactant Tween 80 and spray-drying through high pressure homogenize to obtain nano-turmeric powder with a diameter of 100-1000 nanometers;
the kudzu isoflavone powder is prepared by providing Pueraria root to grind into coarse powder and add distilled water of 5-10 times of the weight of the Pueraria root to stir for 0.5-1 hour and filter to obtain filtrate, repeating the providing Primula sieboldii root for five times to combine the obtained filtrate, letting stand the combined filtrate for 1-2 days at 25-30° C. to obtain a precipitate, and filtering and drying the obtained precipitate to grind into fine powder as the kudzu isoflavone powder;
the Primula sieboldii root powder is prepared by providing Primula sieboldii root to grind into coarse powder and put into a juicer with 100-200 meshes to squeeze to obtain Primula sieboldii root juice, drying the Primula sieboldii root juice in a drying box at 60-80° C. to obtain a dried solid, and putting the dried solid in a pulverizer for pulverizing as the Primula sieboldii root powder;
the fruit extract is prepared by providing fresh fruit to wash and chop and then add ethanol aqueous solution of 5-10 times of the weight of the fruit and mass fraction of 70-80% to fully stir and add pectinase with mass fraction of 0.05% to perform enzymatic hydrolysis for 5-8 hours in 40-50° C. to obtain a enzymatic hydrolysis solution, and heating the enzymatic hydrolysis solution to 100° C. for inactivating the pectinase and centrifuging at 5000 rpm in 25° C. for 20 minutes to obtain a supernatant, drying the obtained supernatant at 30-40° C. to freeze-dry as the fruit extract; and
the vitamin complex is prepared by well mixing at least two of sodium L-ascorbate, dl-α-tocopheryl acetate, pyridoxine hydrochloride, riboflavin, thiamine hydrochloride, and thiamine nitrate in a quality ratio of 1:1 as the vitamin complex.

Specifically, the sieving pretreatment includes:
sieving each of the weighed turmeric powder, kudzu isoflavone powder, Primula sieboldii root powder, fruit extract and vitamin complex with a sieve.
Furthermore, a sieve is used for sieving in the sieving pretreatment, and the sieve has 40 or 60 meshes.

Furthermore, the mix process includes:
adjusting a mixing time according to the total weight of the raw materials; and
obtaining the food by pouring the pretreated turmeric powder, kudzu isoflavone powder, Primula sieboldii root powder, fruit extract and vitamin complex into a mixing unit of the wet granulator for mixing.

Furthermore, the testing process includes:
observing whether the food contains any clump, and determining the food as needing re-performing the mix process in response to the food containing any clump; and
determining the mixed food as qualified for posting with a material label and putting into a warehouse for inspection and releasing, in response to the mixed food not containing any clump.

### Embodiment One

In embodiment one, the producing of 100 g of the food for hangover and liver protection is taken as an example.

The food for hangover and liver protection is made with 25% of turmeric powder, 40% of kudzu isoflavone powder, 28% of Primula sieboldii root powder, 5% of fruit extract, and 2% of vitamin complex.

Furthermore, the turmeric powder is prepared by providing turmeric to grind into coarse powder and add ethanol aqueous solution of 5-8 times of the weight of the turmeric and mass fraction of 70-80% to stir and let stand for 48 hours, providing a supernatant to vacuum concentrate to a saturated solution and let stand for cooling so as to precipitate a solid, and adding the solid after drying to an aqueous solution containing the surfactant Tween 80 and spray-drying through high pressure homogenize to obtain nano-turmeric powder with a diameter of 100-1000 nanometers;
the kudzu isoflavone powder is prepared by providing Pueraria root to grind into coarse powder and add distilled water of 5-10 times of the weight of the Pueraria root to stir for 0.5-1 hour and filter to obtain filtrate, repeating the providing Primula sieboldii root for five times to combine the obtained filtrate, letting stand the combined filtrate for 1-2 days at 25-30° C. to obtain a precipitate, and filtering and drying the obtained precipitate to grind into fine powder as the kudzu isoflavone powder; and
the Primula sieboldii root powder is prepared by providing Primula sieboldii root to grind into coarse powder and put into a juicer with 100-200 meshes to squeeze to obtain Primula sieboldii root juice, drying the Primula sieboldii root juice in a drying box at 60-80° C. to obtain a dried solid, and putting the dried solid in a pulverizer for pulverizing as the Primula sieboldii root powder;
the fruit extract is prepared by providing fresh fruit to wash and chop and then add ethanol aqueous solution of 5-10 times of the weight of the fruit and mass fraction of 70-80% to fully stir and add pectinase with mass fraction of 0.05% to perform enzymatic hydrolysis for 5-8 hours in 40-50° C. to obtain a enzymatic hydrolysis solution, and heating the enzymatic hydrolysis solution to 100° C. for inactivating the pectinase and centrifuging at 5000 rpm in 25° C. for 20 minutes to obtain a supernatant, drying the obtained supernatant at 30-40° C. to freeze-dry as the fruit extract;
the vitamin complex is prepared by well mixing at least two of sodium L-ascorbate, dl-α-tocopheryl acetate, pyridoxine hydrochloride, riboflavin, thiamine hydrochloride, and thiamine nitrate in a quality ratio of 1:1 as the vitamin complex;
weighing and providing 25 g of turmeric powder, 40 g of kudzu isoflavone powder, 28 g of Primula sieboldii root powder, 5 g of fruit extract, and 2 g of vitamin complex; and
sieving each of the weighed turmeric powder, kudzu isoflavone powder, Primula sieboldii root powder, fruit extract and vitamin complex with a sieve.

Furthermore, the pretreated turmeric powder ,the kudzu isoflavone powder, the Primula sieboldii root powder, and the vitamin complex use the sieve with 40 meshes, and the fruit extract use the sieve with 60 meshes;
adjusting the working time of the mixing unit of the wet granulator to 10 minutes;
obtaining the food by pouring the filtered turmeric powder, kudzu isoflavone powder, Primula sieboldii root powder, fruit extract and vitamin complex into a mixing unit of the wet granulator for mixing;
observing whether the food contains any clump, and determining the food as needing re-performing the mix process in response to the food containing any clump; and
determining the mixed food as qualified for posting with a material label and putting into a warehouse for inspection and releasing, in response to the mixed food not containing any clump.

### Embodiment Two

In embodiment two, the producing of 100 g of the food for hangover and liver protection is taken as an example.

The food for hangover and liver protection is made with 25% of turmeric powder, 30% of kudzu isoflavone powder, 30% of Primula sieboldii root powder, 14% of fruit extract, and 1% of vitamin complex.

The providing methods of the turmeric powder, the kudzu isoflavone powder, the Primula sieboldii root powder, the fruit extract, and the vitamin complex are the same as in Embodiment 1.

25 g of turmeric powder, 30 g of kudzu isoflavone powder, 30 g of Primula sieboldii root powder, 14 g of fruit extract, and 1 g of vitamin complex are weighed and provided.

sieving each of the weighed turmeric powder, kudzu isoflavone powder, Primula sieboldii root powder, fruit extract and vitamin complex with a sieve.

Furthermore, the pretreated turmeric powder ,the kudzu isoflavone powder, the Primula sieboldii root powder, and the vitamin complex use the sieve with 40 meshes, and the fruit extract use the sieve with 60 meshes;
adjusting the working time of the mixing unit of the wet granulator to 10 minutes;
obtaining the food by pouring the filtered turmeric powder, kudzu isoflavone powder, Primula sieboldii root powder, fruit extract and vitamin complex into a mixing unit of the wet granulator for mixing;
observing whether the food contains any clump, and determining the food as needing re-performing the mix process in response to the food containing any clump; and
determining the mixed food as qualified for posting with a material label and putting into a warehouse for inspection and releasing, in response to the mixed food not containing any clump.

### Embodiment Three

In embodiment three, the producing of 100 g of the food for hangover and liver protection is taken as an example.

The food for hangover and liver protection is made with 35% of turmeric powder, 38% of kudzu isoflavone powder, 20% of Primula sieboldii root powder, 5% of fruit extract, and 2% of vitamin complex.

The providing methods of the turmeric powder, the kudzu isoflavone powder, the Primula sieboldii root powder, the fruit extract, and the vitamin complex are the same as in Embodiment 1.

35 g of turmeric powder, 38 g of kudzu isoflavone powder, 20 g of Primula sieboldii root powder, 5 g of fruit extract, and 2 g of vitamin complex are weighed and provided.

sieving each of the weighed turmeric powder, kudzu isoflavone powder, Primula sieboldii root powder, fruit extract and vitamin complex with a sieve.

Furthermore, the pretreated turmeric powder, the kudzu isoflavone powder, the Primula sieboldii root powder, and the vitamin complex use the sieve with 40 meshes, and the fruit extract use the sieve with 60 meshes;
adjusting the working time of the mixing unit of the wet granulator to 10 minutes;
obtaining the food by pouring the filtered turmeric powder, kudzu isoflavone powder, Primula sieboldii root powder, fruit extract and vitamin complex into a mixing unit of the wet granulator for mixing;
observing whether the food contains any clump, and determining the food as needing re-performing the mix process in response to the food containing any clump; and
determining the mixed food as qualified for posting with a material label and putting into a warehouse for inspection and releasing, in response to the mixed food not containing any clump.

### Experiments

### Experimental Example 1

Alcohol dehydrogenase activation test

Test sample groups:
group A: water extract of turmeric powder;
group B: water extract of kudzu isoflavone powder;
group C: water extract of Primula sieboldii root powder;
group D: water extract of turmeric powder: water extract of kudzu isoflavone powder =1:1;
group E: water extract of turmeric powder: water extract of Primula sieboldii root powder =1:1;
group F: water extract of kudzu isoflavone powder: water extract of Primula sieboldii root powder =1:1;
group G: water extract of turmeric powder: water extract of kudzu root isoflavone powder: water extract of kudzu isoflavone powder = 1:1:1; and
blank control group: distilled water.

The standard Valle-Hoch method is used to measure the activity of the in vitro alcohol dehydrogenase of each of the above-mentioned test sample groups. 1.5 mL 32 mmol/L of sodium pyrophosphate buffer (PH 9), 0.5 mL 11.5% (v/v) of ethanol solution, 1.0 mL 27 mmol/L of oxidized coenzyme I (NAD+) solution and 0.1 mL of the sample group to be tested are well mixed and keeping warm in a water bath at 25° C. for 15 minutes, and then immediately add 0.1 mL 0.55 ug/mL of alcohol dehydrogenase (ADH) to measure the absorbance value (A₃₄₀ₙₘ) on a spectrophotometer at a wavelength of 340 nm after shaking well. The absorbance value read at every 10 seconds in 5 minutes, and the activation rate of alcohol dehydrogenase is calculated according to the following formula so as to record in Table 1:
the calculation of the activity of alcohol dehydrogenase: calculating the activity of the alcohol dehydrogenase of each of the test sample groups according to the molar extinction coefficient 6.2 at 340 nm; and
the activation rate (%) of alcohol dehydrogenase=(enzyme activity ₜₑₛₜ _{group}-enzyme activity _{blank} _{control} _{group})/enzyme activity _{blank} _{control} _{group}×100%

TABLE 1

the activation rate of alcohol dehydrogenase

| Test group | A | B | C | D | E | F | G | Blank control |
|---|---|---|---|---|---|---|---|---|
| Activation rate (%) | 36.43 | 21.98 | 0.07 | 66.42 | 40.79 | 26.58 | 77.67 | 0 |

As can be seen from the data in Table 1, the turmeric powder and kudzu isoflavone powder in the compositions of the food for hangover and liver protection provided by the present disclosure has a strong promoting effect on the activation of alcohol dehydrogenase, and the activation rate can reach 36.43% and 21.98%, respectively, while the Primula sieboldii root powder has almost no promoting effect on the activation of alcohol dehydrogenase. But when these compositions are used in combination, the activation rate of alcohol dehydrogenase is significantly improved in comparison with using single composition, which indicates that the combined use of a plurality of compositions in the present disclosure has a synergistic improvement effect on activating alcohol dehydrogenase. In addition, when the three compositions are used at the same time, the synergistic effect is the strongest. The activation rate can be as high as 77.67%.

### Experimental Example 2

### Acetaldehyde Dehydrogenase Activation Test

Test sample groups:
group A: water extract of turmeric powder;
group B: water extract of kudzu isoflavone powder;
group C: water extract of Primula sieboldii root powder;
group D: water extract of turmeric powder: water extract of kudzu isoflavone powder = 1:1;
group E: water extract of turmeric powder: water extract of Primula sieboldii root powder = 1:1;
group F: water extract of kudzu powder: water extract of Primula sieboldii root powder = 1:1;
group G: water extract of turmeric powder: water extract of kudzu root isoflavone powder: water extract of kudzu isoflavone powder = 1:1:1; and
blank control group: distilled water.

The standard Blair&Bodley method is used to measure the activity of the vitro acetaldehyde dehydrogenase of each of the above-mentioned test sample groups. 1.5 mL 100 mmol/L of sodium pyrophosphate buffer (PH 10), 0.5 mL 20% (v/v) of acetaldehyde solution, 1.0 mL 3.6 mmol/L of oxidized coenzyme I (NAD+) solution and 0.1 mL of the sample group to be tested are well mixed and keeping warm in a water bath at 30° C. for 5 minutes, and then immediately add 0.1 mL 18 mmol/L of acetaldehyde dehydrogenase (ALDH) to measure the absorbance value (A₃₄₀ₙₘ) on a spectrophotometer at a wavelength of 340 nm after shaking well. The absorbance value read at every 10 seconds until the absorbance value per minute is stable, and the activation rate of acetaldehyde dehydrogenase is calculated according to the following formula so as to record in Table 2:
the calculation of the activity of acetaldehyde dehydrogenase: calculating the activity of the acetaldehyde dehydrogenase of each of the test sample groups according to the molar extinction coefficient 6.2 at 340 nm; and
the activation rate (%) of acetaldehyde dehydrogenase =(enzyme activity ₜₑₛₜ _{group}-enzyme activity _{blank} _{control} _{group})/enzyme activity _{blank} _{control} _{group}×100%

TABLE 2

the activation rate of acetaldehyde dehydrogenase

| Test group | A | B | C | D | E | F | G | Blank control |
|---|---|---|---|---|---|---|---|---|
| Activation rate (%) | 22.97 | 18.03 | 0.02 | 45.88 | 30.76 | 28.09 | 60.12 | 0 |

As can be seen from the data in Table 2, the turmeric powder and kudzu isoflavone powder in the compositions of the food for hangover and liver protection provided by the present disclosure has a strong promoting effect on the activation of acetaldehyde dehydrogenase, and the activation rate can reach 22.97% and 18.03%, respectively, while the Primula sieboldii root powder has no promoting effect on the activation of acetaldehyde dehydrogenase. But when these compositions are used in combination, the activation rate of acetaldehyde dehydrogenase is significantly improved in comparison with using single composition, which indicates that the combined use of a plurality of compositions in the present disclosure has a synergistic improvement effect on activating acetaldehyde dehydrogenase. In addition, when the three compositions are used at the same time, the synergistic effect is the strongest. The activation rate can be as high as 60.12%.

### Experimental Example 3

The effect of the food for hangover and liver protection on the activity of acetaldehyde dehydrogenase (ALDH) in mouse liver cells.

Test sample groups:
experimental example group 1: the food for hangover and liver protection produced in Embodiment one;
experimental example group 2: the food for hangover and liver protection produced in Embodiment two;
experimental example group 3: the food for hangover and liver protection produced in Embodiment three;
control group: 56% liquor; and
blank control group: distilled water.

Test method: 50 healthy mice are selected to randomly divide them 5 groups each having 10 mice, that is, experimental example group 1, experimental example group 2, experimental example group 3, control group, and blank control group. Each group was fed the same amount of samples once a day for 3 consecutive days.

Subsequently, the mice are killed, dissected, and the liver tissues are taken out and cut into pieces, and then put in liquid nitrogen for quickly frozen overnight. The next day, taking out of liquid nitrogen, and immediately ground into powder with liquid nitrogen (the tissue was not Freeze-thaw). The lysis solution is added with the ration of 10 mg of the liver tissue to 10 µL of the lysis solution in the acetaldehyde dehydrogenase activity detection kit to mix well, and incubate in an ice bath for 30 minutes while performing strong vortex oscillation for 30 seconds every 10 minutes. Then, centrifuging for 10 minutes at 4° C. and 12000 rpm to get the supernatant, and the activity of the ALDH in mouse liver cells is measured according to the instructions of the test kit, and then the results are recorded in Table 3:

TABLE 3

the effect of the food for hangover and liver protection to the activity of the ALDH in mouse liver cells

| Test group | Example group 1 | Example group 2 | Example group 3 | Control group | Blank control group: |
|---|---|---|---|---|---|
| ALDH activity (µmol/min/mg) | 86.9±0.3 | 79.1±0.5 | 77.2±0.4 | 44.8±0.1 | 67.9±0.2 |

It can be seen from the data in Table 3 that, compared with the blank control group, the ALDH activity of the liver cells of the mice in the control group will be reduced after being fed liquor, and the food for hangover and liver protection in Embodiments one-three of the present disclosure can significantly improve the ALDH activity of the liver cells of the mice, and can promote the metabolism of acetaldehyde in the liver cells of the mice and help protect the liver.

### Experimental Example 4

### Mouse Righting Test

Test sample groups:
experimental example group 1: the food for hangover and liver protection produced in Embodiment one;
experimental example group 2: the food for hangover and liver protection produced in Embodiment two;
experimental example group 3: the food for hangover and liver protection produced in Embodiment three;
blank control group: distilled water.

Test method: the mice are fed with each of the test sample group first and then gavage with 56% liquor. Whether the mouse is drunk or not is based on the disappearance of the righting reflex as an indicator, that is, the mouse is gently placed in the animal cage with its back down, and if the posture is maintained for more than 30 seconds, it is considered that the righting reflex has disappeared, and it is defined as drunk.

40 healthy mice are selected and randomly divided into 4 groups each having 10 mice, that is, blank control group, experimental example group 1, experimental example group 2, and experimental example group 3. After fasting for 12 hours, each group is fed the same amount of test samples. After 30 minutes, 56% liquor at a dose of 0.2 mL/10 g is fed. The drunkenness (disappearance of the righting reflex) and sobering (recovery of the righting reflex) times (start timing after drinking) for each group are recorded, and then the results are recorded in Table 4.

TABLE 4

mouse righting test

| | | |
|---|---|---|
| Group | disappearance time of the righting reflex (drunkenness time) | recovery time of the righting reflex (sobering time) |
| Example group 1 | 40.35±5.32 (min) | 89.18±8.10 (min) |
| Example group 2 | 48.28±4.16 (min) | 83.91±9.18 (min) |
| Example group 3 | 50.76±4.51 (min) | 76.32±8.37 (min) |
| Blank control group | 22.10±7.74 (min) | 200.05±7.752 (min) |

It can be seen from the data in Table 4 that, compared with the blank control group, the food for hangover and liver protection produced in Embodiments one-three of the present disclosure can postpone the disappearance time of the righting reflex to varying degrees and reduce the recovery time of the righting reflex. It shows that the food for hangover and liver protection produced provided by the present disclosure has the effect of delaying drunkenness and anti-alcoholism.

### Experimental Example 5

### Human Hangover Test

50 volunteers are randomly divided into 5 groups each having 10 people, that is, Example group 1, Example group 2, Example group 3, control group, and blank control group. Each group drink 100 mL of 56% liquor. Example group 1 drinks the food for hangover and liver protection produced in Embodiment one; Example group 2 drinks the food for hangover and liver protection produced in Embodiment two; Example group 3 drinks the food for hangover and liver protection produced in Embodiment three; the control group drinks a commercial anti-alcoholic food; and the blank control group does not drink the anti-alcoholic food. After 30 minutes, the alcohol content in the body is tested with an alcohol tester and recorded in Table 5.

TABLE 5

human body hangover test

| Group | Example group 1 | Example group 2 | Example group 3 | Control group | Blank control group |
|---|---|---|---|---|---|
| Drinking volume | 100 mL | 100 mL | 100 mL | 100 mL | 100 mL |
| Body alcohol content (mg/100mL) | 26±1 | 18±2 | 5±1 | 31±3 | 91±2 |

It can be seen from the data in Table 5 that, the food for hangover and liver protection provided by the present disclosure can effectively reduce the alcohol content in the blood after drinking, and has an anti-alcoholic effect. Compared with a certain commercially anti-alcoholic food, the effect of the present disclosure is more significant.

### Experimental Example 6

The effect of the food for hangover and liver protection on the activity of acetaldehyde dehydrogenase (ALDH) in human liver cells.

Test sample groups:
experimental example group 1: the food for hangover and liver protection produced in Embodiment one;
experimental example group 2: the food for hangover and liver protection produced in Embodiment two;
experimental example group 3: the food for hangover and liver protection

produced in Embodiment three;
control group: 56% liquor; and
blank control group: distilled water.

Test method: the human hepatocytes are inoculated in a normal medium containing 10% fetal bovine serum, and cultured at 95% humidity, 5% CO₂, and 37° C. until the cell fusion reached 80%. Subsequently, equal amounts of each test sample group are added to the medium and incubated for 24 hours. The ALDH activity of the human hepatocytes is measured according to the instructions of the ALDH detection kit, and then the results are recorded in Table 6.

TABLE 6

the effects of the food for hangover and liver protection on the ALDH activity of human liver cells

| Group | Example group 1 | Example group 2 | Example group 3 | Control group | Blank control group |
|---|---|---|---|---|---|
| ALDH activity (µmol/min/mg) | 6.31 | 5.98 | 5.77 | 1.12 | 2.39 |

It can be seen from the data in Table 6 that, compared with the blank control group, after the control group treated human liver cells with 56% liquor, the ALDH activity is reduced, while the food for hangover and liver protection provided in Embodiments one-three of the present disclosure can significantly improve the ALDH activity of human liver cells, and can promote the metabolism of acetaldehyde in human liver cells and help protect the liver. Based on the data in Tables 1-6, it can be seen that the food for hangover and liver protection provided by the present disclosure can effectively improve the activities of alcohol dehydrogenase and acetaldehyde dehydrogenase, promote the metabolism of ethanol and acetaldehyde in the human body, delay drunkenness, and effectively hangover while protecting the liver.

Although the present disclosure is described in detail with reference to the above-mentioned embodiments, it should be understood by those skilled in the art that, the technical schemes in each of the above-mentioned embodiments may still be modified, or some of the technical features may be equivalently replaced, while these modifications or replacements do not make the essence of the corresponding technical schemes depart from the spirit and scope of the technical schemes of each of the embodiments of the present disclosure.

What is claimed is:

1. A food for hangover and liver protection, comprising raw materials of particular percentages by weight, wherein the raw materials include:
25%-35% of turmeric powder;
30%-40% of kudzu isoflavone powder;
20%-30% of Primula sieboldii root powder;
5%-14% of fruit extract; and
1%-2% of vitamin complex.

2. The food of claim 1, wherein the turmeric powder is nano turmeric powder.

3. The food of claim 1, wherein the kudzu isoflavone powder comprises one or two of 10% of isoflavone, 20% of isoflavone, and 40% of isoflavone.

4. The food of claim 1, wherein the Primula sieboldii root powder is ground powder of Primula sieboldii root.

5. The food of claim 1, wherein the fruit extract comprises one or more of extracts of apple, guava, persimmon, and citrus peel.

6. The food of claim 1, wherein the vitamin complex comprises at least two of sodium L-ascorbate, dl-α-tocopheryl acetate, pyridoxine hydrochloride, riboflavin, thiamine hydrochloride, and thiamine nitrate.

7. A computer-implemented producing method for a food for hangover and liver protection, comprising:
preparing raw materials of the food, wherein the raw materials include turmeric powder, kudzu isoflavone powder, Primula sieboldii root powder, fruit extract, and vitamin complex;
providing the raw materials of particular percentages by weight, wherein the prepared turmeric powder accounts for 25%-35% of the provided raw materials; the prepared kudzu isoflavone powder accounts for 30%-40% of the provided raw materials; the prepared Primula sieboldii root powder accounts for 20%-30% of the provided raw materials; the prepared fruit extract accounts for 5%-14% of the provided raw materials; and the prepared vitamin complex accounts for 1%-2% of the provided raw materials;
performing a sieving pretreatment on each of the provided raw materials;
putting the pretreated raw materials into a wet granulator to perform a mix process for obtaining the food; and
performing a testing process on the mixed food, and re-performing the mix process on the tested food in response to the tested food being unqualified.

8. The producing method of claim 7, wherein the turmeric powder is prepared by providing turmeric to grind into coarse powder and add ethanol aqueous solution of 5-8 times of the weight of the turmeric and mass fraction of 70-80% to stir and let stand for 48 hours, providing a supernatant to vacuum concentrate to a saturated solution and let stand for cooling so as to precipitate a solid, and adding the solid after drying to an aqueous solution containing the surfactant Tween 80 and spray-drying through high pressure homogenize to obtain nano-turmeric powder with a diameter of 100-1000 nanometers;
the kudzu isoflavone powder is prepared by providing Pueraria root to grind into coarse powder and add distilled water of 5-10 times of the weight of the Pueraria root to stir for 0.5-1 hour and filter to obtain filtrate, repeating the providing Primula sieboldii root for five times to combine the obtained filtrate, letting stand the combined filtrate for 1-2 days at 25-30° C. to obtain a precipitate, and filtering and drying the obtained precipitate to grind into fine powder as the kudzu isoflavone powder; and
the Primula sieboldii root powder is prepared by providing Primula sieboldii root to grind into coarse powder and put into a juicer with 100-200 meshes to squeeze to obtain Primula sieboldii root juice, drying the Primula sieboldii root juice in a drying box at 60-80° C. to obtain a dried solid, and putting the dried solid in a pulverizer for pulverizing as the Primula sieboldii root powder.

9. The producing method of claim 7, wherein the fruit extract is prepared by providing fruit to wash and chop and then add ethanol aqueous solution of 5-10 times of the weight of the fruit and mass fraction of 70-80% to fully stir and add pectinase with mass fraction of 0.05% to perform enzymatic hydrolysis for 5-8 hours in 40-50° C. to obtain a enzymatic hydrolysis solution, and heating the enzymatic hydrolysis solution to 100° C. for inactivating the pectinase and centrifuging at 5000 rpm in 25° C. for 20 minutes to obtain a supernatant, drying the obtained supernatant at 30-40° C. to freeze-dry as the fruit extract.

10. The producing method of claim 7, wherein a sieve is used for sieving in the sieving pretreatment, and the sieve has 40 or 60 meshes:.

11. The producing method of claim 10, wherein the mix process includes:
adjusting a mixing time according to the total weight of the raw materials.

12. The producing method of claim 11, wherein the mix process further includes:
obtaining the food by pouring the pretreated turmeric powder, kudzu isoflavone powder, Primula sieboldii root powder, fruit extract and vitamin complex into a mixing unit of the wet granulator for mixing.

13. The producing method of claim 12, wherein the testing process includes:
observing whether the food contains any clump, and determining the food as needing re-performing the mix process in response to the food containing any clump; and
determining the mixed food as qualified, in response to the mixed food not containing any clump.
